(19) United States
(12) Patent Application Publication
(10) Pub. No.: US 2024/0099629 A1
(43) Pub. Date: Mar. 28, 2024

(54) Device and Method for Detecting a Medically Active Implant Within a Person

(71) Applicant: Neuroloop GmbH, Freiburg (DE)

(72) Inventors: Dennis PLACHTA, Freiburg (DE); Nils KÖNNE, Freiburg (DE)

(21) Appl. No.: 17/769,094

(22) PCT Filed: Sep. 24, 2020
(86) PCT No.: PCT/EP2020/076734

§ 371 Date: Apr. 14, 2022

(30) Foreign Application Priority Data
Oct. 30, 2019 (DE) 10 2019 216 780.2

(51) Int. Cl. A61B 5/28; A61B 5/251; A61B 5/347; A61N 1/37

(52) U.S. Cl. CPC A61B 5/28; A61B 5/251; A61B 5/347; A61N 1/3704; A61N 1/3706

(57) ABSTRACT

The invention is a device and a method for detecting a medically active implant capable of electrical stimulation implanted within a person. Moreover, the invention also relates to the use of the device for determining the operational status of the medically active implant. The device is characterized by components: a) at least two electrodes that are in, or can be brought into, contact with the person, b) an electrical voltage measuring unit which is electrically connected to the at least two electrodes and which is capable of generating electrical voltage timing signals and c) an evaluation unit which is connected to the electrical voltage measuring unit.

### CROSS-REFERENCE TO RELATED APPLICATIONS

This application is a U.S. national phase application filed under 35 U.S.C. § 371 of International Application No. PCT/EP2020/076734, filed Sep. 24, 2020, designating the United States, which claims priority from German Patent Application No. 10 2019 216 780.2, filed Oct. 30, 2019, which are hereby incorporated herein by reference in their entirety.

### BACKGROUND OF THE INVENTION

### Field of the Invention

The invention relates to a device and a method for detecting a medically active implant capable of electrical simulation implanted within a person. Further described is the use of the device for determining the operating status of the medically active implant.

### Description of the Prior Art

Active implants for treating various diseases are being used in ever more areas of medicine. In contrast to purely passive implants that typically have a purely mechanical support and carrying function, active implants comprise electronic components that are usually able to detect electrical signals for supporting organ functions and to generate and apply them locally for stimulation purposes.

The presence of such an active implant, therefore indicates the presence of the corresponding clinical indication. In the case of an emergency situation involving the patient, knowledge of the clinical indication for the implanted active implant can make a change in the emergency treatment necessary. For this reason, it is important that even in situations in which the patient can no longer provide information by himself/herself, the medical personal are made aware of the presence of an active implant.

In accordance with article 18, para. 2 of the European Medical Device Regulation, patients who have a medically active implant are issued an implant card with which the respective implant can be identified. The information about the implant is therefore only available if the patient can still communicate this and/or the card is being carried.

The documents US published Patent Application 2006/0293714A1, U.S. Pat. Nos. 4,291,703A, 5,406,955A and 9,788,756B2 each disclose devices and methods for detecting a medical implant.

### SUMMARY OF THE INVENTION

The invention is a device and a method for detecting a medically active implant capable of electrical simulation implanted within a person, so that in a non-invasive manner quick and reliable determination of whether an active implant is present may be performed without the necessity of communicating with the person, and if there is, what type of implant this involves. More particularly, it should be possible to identify a detected implant from its stimulation pattern and to assess its operational status.

The device according to the invention for detecting the presence in a person of a medically active implant that is capable of electrical stimulation, comprises at least two electrodes that are in, or can be brought into, contact with the person, an electrical voltage measuring unit, which is connected to the at least two electrodes and is able to generate electrical voltage timing signals, as well as an evaluation unit which is connected to the electrical voltage measuring unit and with which it is possible, on the basis of the electrical voltage timing signals, to derive the information proving the presence, as well as the nature or type of an active implant. For this, the evaluation unit comprises a unit for detecting a power spectral density based on the electrical voltage timing signals measured between the at least two electrodes, preferably in the form of a spectrum analyser. Used for this is a unit for determining local maxima within the power spectral density and the so-called peak frequency allocated to each one, as well as a unit for determining the lowest common denominator among the determined peak frequencies. Finally, the evaluation unit comprises a comparator unit which performs a data comparison on the basis of the determined lowest common denominator that corresponds to a stimulation frequency of the medically active implant and information stored in a reference database, and which generates a comparison outcome, as a function of which a signal unit in communication with the comparator unit produces a signal.

The measuring principle forming the basis of the invention, essentially carries out an electrical voltage measurement between at least two predefined skin surface areas of the person, and analysing the obtained electrical voltage signal in terms of time and spectrum. Also suitable in addition to the use of adhesive electrodes that are applicable to the skin of a person, are electrodes that adhere by way of a temporary negative pressure, as are usually used in connection with standard ECG devices.

Appropriate contact areas for the electrical voltage measurements are preferably areas of the head, neck, upper body, groin or leg, more particularly preferably along known equipotential surfaces of the evoked potential of the heart.

The at least two electrodes applied to the person are connected galvanically, that is by wire, with the electrical voltage measuring unit, which is accommodated as a separate component or as an integral part within an assembly, and also comprises an evaluation unit having several components for the spectral investigation and evaluation of the picked up electrical voltage timing signals. The assembly can be designed as a modular insert that is integrated into existing medical diagnosis apparatuses, or as a portable unit for manual field use.

The electrical voltage timing signals which are recorded by the voltage measuring unit are recorded at a sampling frequency fs of at least 100 Hz, which is preferably with sampling frequency of between 1 MHz and 1 GHz, and are preferably stored within a memory unit. Also stored in the memory unit or in a further memory unit in the form of a reference database, is information for a data comparison.

For digital signal processing and storage of the analog voltage timing signals provided by the voltage measuring unit, the analogue voltage timing signals are converted an analog-digital converter into digital signals, which are supplied to the evaluation unit. The individual components are essentially based on at least one of digital signal processors and microcontrollers.

For this, as a first component, the evaluation unit determines a power spectral density that represents the spectral power density of the frequency-related power of the electrical voltage timing signal within the possible frequency range between 0 and fs/2.

After determination of the power spectral density, in a second component, which determines local maxima, the spectral positioning of the local maxima within the power spectral density is determined, which in each case is characterised by the so-called peak frequency. A possible criterion for the determination of local maxima is, for example, that the power spectral density within a certain frequency width, for example 2 Hz, has decreased by, for example, 3 dB around the peak frequency.

Thereafter, from among the previously determined peak frequencies, a third component of the evaluation unit determines the lowest common denominator from at least among most of the determined peak frequencies, that corresponds to a or the stimulation frequency of a medically active implant within the person. The stimulation frequency as well as the amplitudes of all harmonics of the stimulation frequency are stored in the memory unit for further processing and further information access.

If determination of the lowest common denominator among the determined peak frequencies is not possible, this may be taken as an indication that there is no active implant present in the person, or that a present implant is not active or is not functionable. In this case, the unit for determining a lowest common denominator generates a signal, which in an appropriately encoded form can be shown by a signal unit, preferably on a display, in order to inform a doctor or user of the device accordingly.

If, on the other hand, it is possible to determine a lowest common denominator among the determined peak frequencies, this is considered to be an indication of the existence of an active implant that stimulates intracorporeally with the measured stimulation frequency. By means of a further component provided in the assembly, the comparator unit, at least on the basis of the determined stimulation frequency as well as with information stored in a reference database, a data comparison is carried out that results in a comparison outcome, as a function of which an optically, acoustically or haptically perceivable signal which is generated by way of the signal unit, by which a user is not only informed about the presence of an active implant and its operating status but is also informed about its nature or type, and thereby the possible underlying clinical indications. Preferably the signal unit is designed as a display to show alphanumerical characters.

Through suitable categorization of information stored in the reference database, for example by fault-free or faulty statuses, as well as definitions of general or implant-specific tolerance ranges, in the event of the presence of a medically active implant being detected, statements about its operational status can also be made.

Advantageously, in order to improve the reliability of determining the nature or type of the unknown implant, further information, that can be derived from the measured and possibly stored electrical voltage timing signals, is also used and compared by the comparator unit with corresponding information stored in the reference database. Suitable examples of additional information are the sequence of the measured voltage timing signals, as well as gleanable or derivable information about the stimulation properties of the medically active implant, such as the stimulation form, stimulation pulse width, stimulation pulse symmetry, stimulation pulse number, stimulation pulse amplitude and time sequence, as well the time correlation of the stimulation pulses relative to the heartbeat.

From the time course of the power spectral density in the area of the determined stimulation frequency as well as its higher harmonics, characteristic stimulation patterns can also be extracted and compared with suitable reference data. If the active implant is, for example, a cardiac pacemaker, this related stimulation pattern is characterized by short stimulation pulses which are synchronised with the heartbeat. On the other hand, if the active implant is a stimulator for reducing blood pressure, the related stimulation signals are characterised by stimulation signals that tend to appear continuously in terms of time. In a further preferred form of embodiment, the electrical voltage unit is electrically connected to at least three electrodes which are placed on different areas of a person's skin surface. Through the resulting possibility of measuring at least three electrical voltage timing signals, the location of the medically active implant within the person can be determined by way of a triangulation measurement. For this, the evaluation unit comprises a triangulation unit, which on the basis of the electrical voltages measured between the at least three electrodes, carries out localization of the medically active implant.

Preferably, all the components of the evaluation unit, that is the unit for determining the power spectral density, the unit for determining local maxima within the power spectral density, the unit for determining the lowest common denominator, the unit for determining the stimulation pulse properties as well as the comparator unit, are digital processors or microcontrollers which lock in a predefined manner by software-based evaluation and determination regulations.

A further preferred embodiment of the device according to the invention provides for an interface, via which the electrical voltage timing signal from an ECG device can be directly supplied to the evaluation unit and used for the detection and identification of an active implant that may possibly be located within a person. In this case, the electrodes are part of the ECG device. It is also possible that through the appropriate addition of an ECG measuring unit, the device in accordance with the invention takes over the entire measurement and recording of an electrocardiogram of a person so that the detection and identification of a possible active implant on the basis of the ECG voltage timing signals can be carried out while performing standard ECG monitoring.

### BRIEF DESCRIPTION OF THE DRAWINGS

The invention will be described below, without restricting the generalinvention, by way of an example of embodiment with reference to the drawings. In these:

FIG. 1 shows a schematic view of a device according to the invention for the detection and identification of an active medical implant.

### DETAILED DESCRIPTION OF THE INVENTION

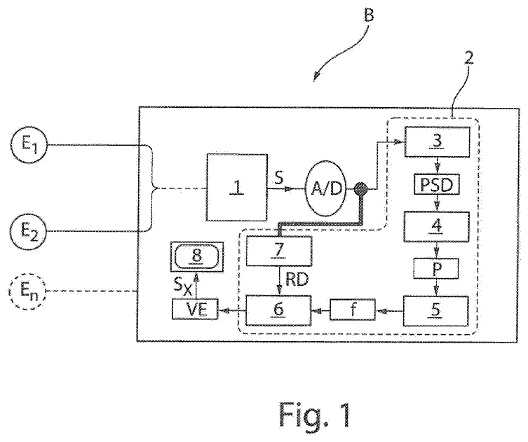

FIG. 1 shows an assembly B, which can be configured as a modular unit inserted into a medicine rack, or as a portably usable functional unit.

The device is connected to at least two electrodes E1, E2, but can be expanded as desired to n electrodes, which are each designed to be firmly attached to the skin surface of a person in a releasable manner.

Provided in the assembly B to record the electrical voltage between the electrodes E1, E2 is a voltage measuring unit 1, which records the electrical potential difference between the electrodes E1, E2 in a time-resolved manner and generates electrical voltage timing signals S. The analog electrical voltage timing signals S are converted by an analog-digital converter A/D into digital voltage timing signals, which for further evaluation or assessment are supplied to both the evaluation unit 2, which is part of the assembly B, and the memory unit (7).

Provided within the evaluation unit 2 is a unit 3 for determining the power spectral density, in which processor-supported spectral analysis of the measured electrical voltage timing signals S is carried out obtaining a power spectral density PSD. In addition, the evaluation unit 2 comprises a unit 4 for determining local maxima within the power spectral density PSD, to each of which is allocated peak frequency P. In a further unit 5, for the obtained or determined peak frequencies P a lowest common denominator, which corresponds to the so-called stimulation frequency f, is determined.

Comparator unit 6 performs a comparison based on reference data RD stored in a memory unit 7 and at least the determined stimulation frequency f is performed in order to obtain a comparison outcome. Optionally, to carry out the comparison, the comparator unit 6 can be provided with additional information which can be extracted from at least one of the electrical voltage timing signals S and/or digitalised voltage timing signals, the pulse form of the stimulation pulses of the medically active implant, their pulse symmetry, pulse width, pulse amplitude, pulse number, pulse timing, and as well as the time correlation with the heartbeat. With regard to the further information, corresponding reference data are stored in the memory unit for comparison.

As a function of the comparison outcome VE, the comparator unit 6 generates a signal SX, that is supplied to a signal unit 8, which depending on the signal SX, appears at least one of visually, acoustically and haptically. Preferably the signal unit 8 is a display, by way of which the information about a detected active implant or its operational status, as well as underlying, possible clinical indications can be shown as alphanumerical characters.

### LIST OF REFERENCE NUMBERS

B Assembly
1 Voltage measuring unit
2 Evaluation unit
3 Unit for determining a power spectral density
4 Unit for determining local maxima within the power spectral density
5 Unit for determining a lowest common denominator of the determined peak frequencies
6 Comparator unit
7 Memory unit
8 Signal unit
PSD power spectral density
P Peak frequency
f Stimulation frequency
RD Reference data
VE Comparison outcome
SX Result signal

1-17. (canceled)

18. A device for detecting in a person a medically active implant configured to provide electrical stimulation, comprising:
at least two electrodes that are configured to or can be brought into contact with the person;
an electrical voltage measuring unit electrically connected to the at least two electrodes which is configured to generate electrical voltage timing signals; and
an evaluation unit connected to the electrical voltage measuring unit including a unit for determining power spectral density based on the electrical voltage timing signals measured between the at least two electrodes;
a unit for determining local maxima within the determined power spectral density and their respective peak frequency of each timing signal;
a unit for determining a lowest common denominator of at least some of the determined peak frequencies;
a comparator which performs a data comparison, based on the determined lowest common denominator corresponding to a stimulation frequency of the medically active implant and information stored in a reference data base, which generates a comparison outcome; and
a signal unit coupled to the comparator which generates a signal as a function of the comparison outcome.

19. The device according to claim 18, wherein:
the electrodes are skin contact electrodes.

20. The device according to claim 18, wherein:
the electrodes are ECG electrodes.

21. The device according to claim 18, wherein:
the unit for determining the power spectral density is a spectrum analyser.

22. The device according to claim 18, wherein for obtaining digitalized electrical voltage timing signals, the electrical voltage measuring unit is connected to an A/D converter with an output connected to the evaluation unit comprising at least one digital signal processor or microcontroller.

23. The device according to claim 22, wherein:
the evaluation unit comprises a memory in which at least the digitalized electrical voltage timing signals are stored.

24. The device according to claim 18, wherein:
the evaluation unit comprises a unit for determining information derivable from the electrical voltage timing signals which relates to stimulation pulses that are generated by the medically active implant, and which contains at least one of information from stimulation pulse form, stimulation pulse symmetry, stimulation pulse width, stimulation pulse amplitude, stimulation pulse number, time sequence of the stimulation pulses, and a time correlation between stimulation pulses and heartbeat.

25. The device according to claim 23, wherein:
the comparator compares at least one of electrical voltage timing signals stored in the memory, and information derived therefrom, with information in the reference database corresponding to at least one of the voltage timing signals and information derived therefrom, from known medical implants.

26. The device according to claim 23, wherein:
the comparator performs a function pattern comparison between at least one of stored voltage timing signals and information derived therefrom, and at least one of voltage timing signals and the information derived therefrom from known medical implants.

27. The device according to claim 18, wherein:
the signal generated by the signal unit is a visually and acoustically perceivable.

28. The device according to claim 18, wherein:
the electrical voltage measuring unit is electrically connected to at least three electrodes, and the evaluation unit comprises a triangulation unit, which based on electrical voltages measured between the at least three electrodes, carries out localization of the medically active implant.

29. A method of use of a device for detecting an unknown active medical implant according to claim 18 to provide identification thereof.

30. A method of use of a device according to claim 18, for determining the operational status of an active medical implant by at least one of comparing at least one of electrical voltage timing signals stored in the memory unit and the information derived therefrom, with categorized information stored in a reference database relating to fault-free or faulty statuses of the implant and definitions of general or specific implant tolerance ranges.

31. A method of use of a device method for detecting a medically active implant present in a person by steps of:
carrying out an electrical voltage measurement by at least two electrodes that are configured to be in electrical contact with a person to order to obtain electrical voltage timing signals;
performing a spectral analysis of measured electrical voltage timing signal to obtain a power spectral density;
determining local maxima within a power spectral density and a peak frequency respectively allocated thereto;
determining a lowest common denominator of determined peak frequencies corresponding to a stimulation frequency of the medically active implant;
comparing the stimulation frequency with reference data to obtain a comparison outcome; and
identifying a medically active implant based on the comparison outcome.

32. The method according to claim 31, wherein:
based on the electrical voltage timing signals, information is derived relating to stimulation pulses produced by the medically active implant and which at least includes information from stimulation pulse form, stimulation pulse symmetry, stimulation pulse width, stimulation pulse amplitude, stimulation pulse number, time sequence of the stimulation pulses, and time correlation between stimulation pulses and heartbeat.

33. The method according to claim 32, wherein:
the information derived from the voltage timing signals is compared with reference data to obtain a comparison outcome used for identifying the medical implant.

34. The method according to claim 32, wherein:
the information derived from the voltage timing signals is compared with reference data to obtain a comparison outcome used to determine operational status of an active medical implant.
